(19) United States
(12) Patent Application Publication
(10) Pub. No.: US 2012/0094315 A1
(43) Pub. Date: Apr. 19, 2012

(54) BIOMARKERS FOR THE DIAGNOSIS AND/OR PREDICTION OF SUSCEPTIBILITY TO MENTAL AND NEURODEGENERATIVE DISORDERS

(76) Inventor: Stephanie Fryar-Williams, Rose Park (AU)

(21) Appl. No.: 13/133,409

(22) PCT Filed: Dec. 9, 2009
(86) PCT No.: PCT/AU2009/001600

§ 371 Date: Dec. 22, 2011

(30) Foreign Application Priority Data
Dec. 9, 2008 (AU) 2008906361
Oct. 16, 2009 (AU) 2009905053

(51) Int. Cl. G01N 33/566; C12Q 1/02; G01N 33/559; C12Q 1/48

(52) U.S. Cl. 435/7.92; 435/15; 435/29; 436/501; 435/7.1; 204/456; 204/464

(57) ABSTRACT

Provided herein are, inter alia, methods for predicting the susceptibility of a subject to a mental or neurodegenerative disorder, the method comprising obtaining one or more biological samples from the subject; determining the levels of one or more biomarkers in the sample, wherein the biomarkers are selected from pyrroles, histamine, methionine adenosyltransferase (MAT) activity, homocysteine, copper and zinc; and comparing the level(s) of the biomarker(s) determined in (b) with the level(s) of said biomarker(s) from one or more control samples, wherein abnormal levels of the one or more biomarkers in the sample(s) from the subject compared to the one or more control samples is predictive of susceptibility of the subject to a mental or neurodegenerative disorder.

### FIELD OF THE INVENTION

The present invention relates to the identification of novel biomarkers and biomarker combinations for the diagnosis of, and prediction of susceptibility to, mental and neurodegenerative disorders and to methods for the diagnosis of, and prediction of susceptibility to, such disorders employing the biomarkers and biomarker combinations. The invention also relates to methods for predicting likely severity and disability associated with mental and neurodegenerative disorders, methods for determining appropriate treatment regimes for sufferers of such disorders as well as to methods for evaluating and monitoring the efficacy and progress of treatment regimes.

### BACKGROUND OF THE INVENTION

Many mental health conditions have their onset between the ages of 18 and 24 years of age and many may appear even earlier in youth. The incidence of mental illness and the impacts of this on society appear to be increasing. Similarly, the burden caused by neurodegenerative conditions on society, the health system, families and individuals increases as the population ages, although it is recognised that neurodegenerative conditions can manifest themselves in younger individuals.

Because of the immense personal, social and financial impact of these conditions on individuals, the community, the health system and the economy, the ability to statistically predict those individuals that are at risk of developing such seriously disabling disorders is of critical importance, as is the ability to accurately and rapidly diagnose mental disorders to ensure early and effective intervention where appropriate.

Current diagnostic approaches typically rely predominantly on symptomatic analyses. For example, diagnosis of a mental or neurodegenerative disorder, or prediction of susceptibility to such a disorder may be made on the basis of one or more of: physical examination; gross medical evaluation; psychological and/or psychiatric evaluation; family history; and emotional history. However in view of the complexity, and often asymptomatic nature, of many mental and neurodegenerative disorders the reliance on symptomatic evaluation and personal history renders diagnosis difficult and often inaccurate. It can be challenging to distinguish between many disorders given common clinical presentation and, moreover, sufferers may remain asymptomatic for years between episodes. Accordingly a long period of extensive observation and evaluation is typically required in order to make a definitive diagnosis.

In assessing which patients require closer follow-up attention, clinicians have in the past relied upon the presence of a cluster of heterogeneous early symptoms, many of which, such as auditory hallucinations that were previously considered a hallmark of psychosis, have now also been shown to be present even in normal populations (see, for example, Van Os et al., 2000). Further, a single occurrence of a positive family history of adult mental illness has not of itself been generally considered a sufficient indicator for close pre-emptive follow up of children in families so-affected by mental illness. More reliable, objective indicators are clearly needed.

Added to the difficulty of deciding which children or young adults should receive close preventative follow-up, has been difficulty with psychiatric classification systems themselves. Unexplained symptom-heterogeneity across diagnoses, increasingly-recognised developmental links with pre-morbid soft neurological signs and emergent adult psychiatric diagnoses, overlapping co-morbid conditions and findings on neuro-imaging are all serving to undermine the clinician's confidence in existing classification systems and provoke concern regarding their validity (Hyman, 2007).

Accordingly, there is a clear need for improved methods for prediction of susceptibility to, and diagnosis of, mental and neurodegenerative disorders and for the improved determination and monitoring of treatments for such disorders to improve disease management and ensure that patients receive the most efficacious treatment.

### SUMMARY OF THE INVENTION

The present invention is predicated on the inventor's surprising findings that blood and urine levels of a range of biochemical markers have predictive value for the diagnosis of mental and neurodegenerative disorders, based on the dysregulation of biomarker levels in patients suffering from a mental disorder relative to normal endogenous levels of such markers.

According to a first aspect the present invention provides a method for predicting the susceptibility of a subject to a mental or neurodegenerative disorder, the method comprising:

(a) obtaining one or more biological samples from the subject;
(b) determining the levels of one or more biomarkers in the sample(s), wherein the biomarkers are selected from pyrroles, histamine, MAT activity, homocysteine, copper and zinc; and
(c) comparing the level(s) of the biomarker(s) determined in (b) with the level(s) of said biomarker(s) from one or more control samples,
wherein abnormal levels of the one or more biomarkers in the sample(s) from the subject compared to the one or more control samples is predictive of susceptibility of the subject to a mental or neurodegenerative disorder.

The biological sample may be derived from any suitable body fluid or tissue. For example the sample may comprise blood (such as erythrocytes, leukocytes, whole blood, blood plasma or blood serum), saliva, sputum, urine, breath, condensed breath, amniotic fluid, cerebrospinal fluid or tissue (post-mortem or living, fresh or frozen). In a particular embodiment the sample comprises whole blood, blood serum or urine.

In a particular embodiment, the method comprises obtaining a blood sample and a urine sample from the subject and determining the levels of each of the biomarkers from one or both of the blood and urine samples.

Typically the control sample is derived from one or more individuals known not to suffer from a mental or neurodegenerative disorder or alternatively known to suffer from a specific, diagnosed mental or neurodegenerative disorder. Where the method comprises obtaining and determining the levels of biomarkers from a blood and a urine sample from the subject, the control samples will also typically comprise blood and urine samples.

Typically the pyrroles are determined from a urine sample (urinary pyrroles). In an embodiment, the pyrrole determined is hydroxyhemopyrrolene-2-one (HPL).

Typically the histamine is serum histamine, optionally as a surrogate methylation activity marker. Typically the methylation activity represents the activity of the enzyme methionine adenosyltransferase (MAT) or an isoenzyme thereof. MAT activity may be measured directly or indirectly. The measurement may be measurement of the levels of one or more products of MAT, S-adenosylmethionine (SAMe or AdoMet) levels and/or S-adenosylhomocysteine (SAH), or the ratio of S-adenosylmethionine to S-adenosylhomocysteine (SAM:SAH ratio).

Typically the homocysteine is plasma homocysteine, for example fasting plasma homocysteine levels.

Typically the copper is free copper, more typically free serum copper. Serum copper and serum ceruloplasmin levels may be determined to calculate free serum copper.

Typically the zinc is plasma zinc.

In an embodiment, the ratio of plasma zinc to free serum copper is also determined.

In an embodiment, abnormal levels of two or more biomarkers in the sample from the subject compared to the one or more control samples is indicative of susceptibility of the subject to a mental or neurodegenerative disorder. In an embodiment, abnormal levels of three or more biomarkers in the sample from the subject compared to the one or more control samples is indicative of susceptibility of the subject to a mental or neurodegenerative disorder.

According to a second aspect the present invention provides a method for diagnosing a mental or neurodegenerative disorder in a subject, the method comprising:

(a) obtaining one or more biological samples from the subject;
(b) determining the levels of one or more biomarkers in the sample(s), wherein the biomarkers are selected from pyrroles, histamine, MAT activity, homocysteine, copper and zinc; and
(c) comparing the level(s) of the biomarker(s) determined in (b) with the level(s) of said biomarker(s) from one or more control samples,
wherein abnormal levels of the one or more biomarkers in the sample(s) from the subject compared to the one or more control samples is indicative of a mental or neurodegenerative disorder in the subject.

In an embodiment, abnormal levels of two or more biomarkers in the sample from the subject compared to the one or more control samples is indicative of a mental or neurodegenerative disorder. In an embodiment, abnormal levels of three or more biomarkers in the sample from the subject compared to the one or more control samples is indicative of a mental or neurodegenerative disorder.

In accordance with the second aspect the method may be used to diagnose the phenotype or endophenotype of a mental or neurodegenerative disorder.

Similarly, in accordance with the second aspect the method may be used to determine or predict the severity and/or disability associated with a mental or neurodegenerative disorder. The subject may or may not be suffering from a mental or neurodegenerative disorder. Typically in accordance with this embodiment the method comprises the step of correlating the number of abnormal biomarker levels with severity and/or disability scores obtained from a predetermined set of patient norms.

By way of example, in accordance with an embodiment of the first or second aspect, elevated urinary pyrrole levels in a biological sample derived from a subject correlate with severity and disability scores and may be indicative of a mental disorder, such as schizophrenia, bipolar disorder, or developmental delay disorders, or symptoms associated therewith, or susceptibility thereto.

By way of example, in accordance with an embodiment of the first or second aspect, reduced serum homocysteine levels in a biological sample derived from a subject may be indicative of a mental disorder, such as depression, a developmental delay disorder, a behaviour disorder, autism, a central auditory processing disorder or an anxiety disorder, or symptoms associated therewith, or susceptibility thereto.

By way of example, in accordance with an embodiment of the first or second aspect a reduced histamine level may be indicative of psychotic symptoms or psychotic disorder or symptoms associated therewith, or susceptibility thereto.

By way of example, in accordance with an embodiment of the first and second aspect an elevated histamine level may be indicative of enhanced distractibility and hypoactivity or symptoms associated therewith, or susceptibility to.

By way of example, in accordance with an embodiment of the first or second aspect, a low plasma zinc to free serum copper ratio may be indicative of depression or symptoms associated therewith, or susceptibility thereto.

By way of example, in accordance with an embodiment of the first or second aspect, accrual of abnormal levels of three of more of said biomarkers may be indicative of central auditory processing disorder, schizophrenia or depression, or other mental illness, personality or behaviour disorder symptoms associated therewith, or susceptibility thereto.

The method in accordance with the first or second aspect may further comprise in step (b) the determination of levels of one or more additional biomarkers as disclosed herein.

A third aspect of the invention provides a method for predicting the onset of a mental or neurodegenerative disorder, or one or more symptoms associated therewith, in a subject, the method comprising:

(a) obtaining one or more biological samples from the subject;
(b) determining the levels of a panel of biomarkers in the sample(s), the biomarkers including pyrroles, histamine, MAT activity, homocysteine, copper and zinc; and
(c) comparing the levels of the biomarkers determined in (b) with the levels of said biomarkers from one or more control samples,
wherein abnormal levels of at least three of the biomarkers in the sample(s) from the subject compared to the one or more control samples is predictive of the onset of a mental or neurodegenerative disorder or one or more symptoms associated therewith in the subject.

A fourth aspect of the invention provides the use of a panel of biomarkers for the diagnosis of a mental or neurodegenerative disorder in a subject, for predicting the susceptibility of a subject to a mental or neurodegenerative disorder and/or predicting the onset of a mental disorder, or one or more symptoms associated therewith, in a subject, wherein the panel of biomarkers comprises pyrroles, histamine, MAT activity, homocysteine, copper and zinc.

The panel of biomarkers may further comprise one or more additional biomarkers as disclosed herein.

A fifth aspect of the invention provides a panel of biomarkers for the diagnosis of a mental or neurodegenerative disorder in a subject, for predicting the susceptibility of a subject to a mental or neurodegenerative disorder and/or predicting the onset of a mental or neurodegenerative disorder, or one or more symptoms associated therewith, in a subject, wherein the panel of biomarkers comprises pyrroles, histamine, MAT activity, homocysteine, copper and zinc.

A sixth aspect of the invention provides a method for determining disease control in a subject suffering from a mental or neurodegenerative disorder, the method comprising:

(a) obtaining one or more biological samples from the subject;
(b) determining the levels of a panel of biomarkers in the sample(s), the biomarkers including pyrroles, histamine, MAT activity, homocysteine, copper and zinc; and
(c) comparing the levels of the biomarkers determined in (b) with the levels of said biomarkers from one or more control samples,
wherein the levels of at least three of the biomarkers in the sample(s) from the subject compared to the one or more control samples is indicative of disease control in the subject.

The method may further comprise monitoring disease control over time in the subject comprising: repeating steps (a) and (b) at least once over a period of time; and determining whether the biomarker levels change over the period of time.

In an embodiment, a reduction in urinary pyrrole levels may be indicative of an improvement in disease control. In an embodiment, an increase in plasma homocysteine levels may be indicative of an improvement in disease control. In an embodiment, a return of free copper or zinc levels or ratios thereof to normal levels may be indicative of an improvement in disease control.

Disease control may be improved by adjusting the timing, frequency and/or intensity of biomarker testing and/or adjusting the identity, timing and/or intensity of a treatment regime to thereby normalise the levels of one or more of the biomarkers.

A seventh aspect of the invention provides a method for evaluating the efficacy of a treatment regime in a subject suffering from a mental or neurodegenerative disorder, the method comprising:

(a) treating the subject with a treatment regime for a period sufficient to evaluate the efficacy of the regime;
(b) obtaining one or more biological samples from the subject;
(c) determining the levels of a panel of biomarkers in the sample(s) in accordance with any one of the above aspects;
(d) repeating steps (b) and (c) at least once over a period of time; and
(e) determining whether the biomarker levels changes over the period of time,
wherein a change in the level of one or more of the biomarkers is indicative of a change in disease control in the subject and the degree of efficacy of the treatment regime.

The treatment regime may comprise the administration of one or more drugs, medications or supplements or one or more forms of psychological or social intervention aimed at directly treating the disorder, early intervention for the disorder, management of residual symptoms of the disorder, prevention of relapse, or overcoming treatment resistance in the subject.

An eighth aspect of the invention provides a method for designing a suitable treatment regime for a subject suffering from a mental or neurodegenerative disorder, the method comprising monitoring the levels of a panel of biomarkers in the subject in accordance to any one of the above aspects in the presence or absence of a treatment regime for treating the mental or neurodegenerative disorder and adjusting the identity, timing and/or intensity of the treatment regime so as to normalise the levels of one or more of the biomarkers.

A ninth aspect of the invention provides a method for treating a subject suffering from a mental or neurodegenerative disorder comprising administering to the subject a treatment regime designed in accordance with the eighth aspect.

A tenth aspect of the invention provides a method for identifying a compound suitable for treating a mental or neurodegenerative disorder, the method comprising the steps of:
(a) isolating at least one cell from a subject suffering from a mental or neurodegenerative illness;
(b) determining the levels of a panel of biomarkers in accordance with any one of the above aspects;
(c) contacting the at least one cell with a candidate compound; and
(d) determining the levels of a panel of biomarkers in accordance with any one of the above aspects,
   wherein normalisation of the levels of one or more of the biomarkers between steps (b) and (d) is indicative of the ability of compound to treat the mental or neurodegenerative disorder.

Methods embodied by the above aspects and embodiments of the invention are particularly suitable for diagnosing and evaluating the status of mental and neurodegenerative disorders in human subjects. However, the invention is not limited thereto and extends to any mammal, for example mammals useful as a model for mental and neurodegenerative disorders in humans. Typically the subject is a mammal, more typically a human. The subject may be of any age, child, adolescent, adult or elderly.

### BRIEF DESCRIPTION OF THE DRAWINGS

The invention will now be described by way of non-limiting example only, with reference to the accompanying drawings.

FIG. 1: Distribution of diagnosed mental disorders and distribution of symptom groups in patients recruited for the present study. CAPD, Central Auditory Processing Disorder; DEPN, Depression; SOMAT, Somatoform Disorder; SCZ, Schizophrenia; ODD, Oppositional Defiance Disorder; AUTISM, Autism; ADD, Attention Deficit Disorder; OCD, Obsessive Compulsive Disorder; ASP, Aspergers Disorder; BPAD, Bipolar Affective Disorder; CD, Conduct Disorder; ADHD, Attention Deficit Hyperactivity Disorder; GAD, Generalised Anxiety Disorder; ID, Intellectual Delay; DOWNS, Downs Syndrome; TOUR, Tourette Syndrome; ANOREX, Anorexia; SCZ-AFF, Schizoaffective Disorder.

FIG. 2: Number and distribution of abnormal biomarkers amongst patients (A) and controls (B). The statistical significance of the difference between patients and controls for three abnormal markers was P=0.001. The statistical significance of the difference between patients and controls for four abnormal markers was P=0.067.

FIG. 3: Comparison of abnormal levels in patients and controls of urinary pyrroles measured as HPL (A), serum homocysteine (B), serum histamine (C), plasma zinc (D), free serum copper (E), and plasma zinc to free serum copper ratio (F).

### DETAILED DESCRIPTION OF THE INVENTION

The articles “a” and “an” are used herein to refer to one or to more than one (i.e., to at least one) of the grammatical object of the article. By way of example, “an element” means one element or more than one element.

Throughout this specification and the claims which follow, unless the context requires otherwise, the word “comprise”, and variations such as “comprises” or “comprising”, will be understood to imply the inclusion of a stated element, integer or step, or group of elements, integers or steps, but not the exclusion of any other element, integer or step, or group of elements, integers or steps.

The term “biomarker” as used herein refers to a naturally occurring, biologically functional compound or molecule that has predictive value for the diagnosis of a mental or neurodegenerative disorder. Determination of the level or activity of a marker in a biological sample may comprise the detection and quantitation of the marker itself or of a precursor, derivative or metabolite thereof. The terms “biomarker” and “marker” may be used interchangeably herein.

As used herein, the term “disability” refers to the degree of adverse impact experienced, ascertained, formally-assessed or reported by a symptomatic subject with a mental or neurodegenerative disorder in carrying out the physical, cognitive and emotional functions of everyday life.

As used herein, the term “disease control” means the status of the disease or disorder, typically in light of intervention to treat the disease or disorder. Thus “disease control” describes the range and severity of symptoms and conditions experienced and suffered by patients as a result of their mental disorder. Disease control effectively provides a measure at a given point in time of the disease status of an individual, reflecting both current therapeutic treatment regimes used by the individual and the individual's recent experiences and psychological state.

As used herein the term “mental disorder” refers generally to mild and severe mental illness health conditions, such as psychiatric or neuropsychiatric diseases, mood disorders, psychotic disorders, personality disorders, pre- and post-traumatic stress disorders, anxiety disorders, developmental disorders, learning disorders, sensory processing disorders, movement disorders, memory disorders, and behavioural disorders as well as other mental disorders and diseases. The disorder or condition may be one that requires or is amenable to intervention in the form of either drug administration or other medical, psychological or psychiatric treatment, but this need not be the case.

As used herein the term “neurodegenerative disorder” refers to any disorder, disease or condition associated with the progressive degeneration of neuron function and/or integrity of neurons in the central or peripheral nervous systems.

For both “mental” and “neurodegenerative” disorders, as these terms are defined and used herein, the disorder may be a disorder classified according to conventional psychiatric guidelines and/or classification schemes of the art (such as DSM IV or DSM IV-R) or alternatively may be defined by a set of symptoms as defined herein below.

The terms “disorder”, condition” and “disease” are used interchangeably herein. Similarly, the term “mental illness” as used herein also refers generally to “mental” and “neurodegenerative” diseases as defined herein.

As used herein, the terms “phenotype” and “endophenotype” refer to the outward, physical manifestation or any discerned or observable characteristic, state or trait of a mental or neurodegenerative disorder, such as morphology, development, biochemical or physiological properties, or behaviour.

As used herein, the term “pyrrole” refers to a pyrrole or pyrrole-related compound including, for example, kryptopyrroles, hydroxyhemopyrrolene-2-one (HPL); and 2,4-dimethyl-3-ethylpyrrole. HPL is referred to in the art by a variety of synonyms including hydroxyhemopyrrole lactam and 3-ethyl-5-hydroxy-4,5-dimethyl-delta3-pyrroline-2-one. All synonyms of HPL are encompassed by the present disclosure. Pyrrole levels may be measured in any body fluid or tissue, for example urine (“urinary pyrroles” and “urinary kryptopyrroles”).

As used herein, the term “severity” refers to the degree of symptom intensity experienced, ascertained, formally assessed or reported by a symptomatic subject with a mental or neurodegenerative disorder.

Reference to “susceptibility” should be understood as a reference to both determining whether any existing events or symptoms associated with or indicative of a mental or neurodegenerative disorder experienced by an individual are linked to abnormal biomarker levels as described herein and to determining whether individuals who have not experienced events or symptoms indicative of a mental or neurodegenerative disorder nevertheless exhibit a predisposition or risk thereto. Thus, depending on the particular circumstances of a particular subject, the term “susceptibility” should be understood to mean vulnerability to a mental or neurodegenerative disorder or having an increased likelihood of development of a mental or neurodegenerative disorder in the future.

As used herein the term “treatment”, including variations thereof, refers to any and all treatments which remedy a disorder or one or more symptoms of a disorder, prevent the establishment of a disorder, provide early intervention for a disorder, provide management of residual symptoms of a disorder, prevent relapse of a disorder, overcome treatment resistance in a disorder, or otherwise prevent, hinder, retard, or reverse the progression of, or other undesirable symptoms of, a disorder in any way whatsoever. Thus the term “treatment” is to be considered in its broadest context. For example, treatment does not necessarily imply that a patient is treated until total recovery. Rather, “treatment” encompasses reducing the severity of, or delaying the onset of, a particular disorder. In the context of some disorders, methods of the present invention involve “treating” the disorder in terms of reducing or ameliorating the occurrence of a highly undesirable event associated with the disorder or an irreversible outcome of the progression of the disorder but may not of itself prevent the initial occurrence of the event or outcome. Accordingly, treatment includes amelioration of the symptoms of a particular disorder or preventing or otherwise reducing the risk of developing a particular disorder.

As disclosed herein, the inventor has surprisingly found that the urine and/or serum levels of a panel of biochemical markers correlates with mental disorder diagnosis. As exemplified herein, disregulation of urinary pyrrole (measured as HPL) levels, serum histamine levels, serum homocysteine levels, serum free copper levels, serum zinc levels and the plasma zinc to free serum copper ratio, when compared to normal endogenous levels of these biomarkers are indicative of mental illness. More particularly, as exemplified herein the distribution of abnormal biomarkers for symptomatic participants was significantly different than for asymptomatic controls (P=0.002), with participants who accrued 3 or more abnormal markers (n=51, P=<0.001), significantly attracting a psychiatric diagnosis.

Thus, disclosed herein for the first time is a simple biochemical test that facilitates the diagnosis, assessment and monitoring of mental or neurodegenerative disorders, prediction of susceptibility, determination or prediction of disease phenotype, severity and/or disability, the identification of appropriate therapeutic treatment regimes for individual patients, and the assessment and monitoring of the effectiveness of existing treatments. The inventor's novel findings as disclosed and exemplified herein open the way for the development of an accurate, cost effective, rapid alternative to presently available methodology for diagnosing, further assessing and monitoring mental and neurodegenerative disorders and monitoring the status of a disorder in affected individuals. The development of a blood and urine-based diagnostic test offers the ability to greatly speed up the diagnostic process and enable accurate differential diagnosis between disorders. A suitable diagnostic test also offers the ability to understand diagnosis and phenotype, predict risk of occurrence/recurrence, severity, disability and treatment-response, and also to expand present disease assessment in order to overcome treatment-resistance or chronicity in patients. The prediction of susceptibility, and risk of occurrence or recurrence enables early intervention and administration of an effective therapies and treatment regimes. Early and effective treatment can improve prognosis and reduce the chance of recurrence of symptoms. Similarly, the prediction of risk of severity and disability enables early intervention and administration of effective medication therapies and psycho-social treatment regimes. Early and effective treatment can improve prognosis and reduce the chance of poor outcome and consequent disability.

By measuring biomarker levels in accordance with embodiments as disclosed herein, a mental health index can be determined. For example the mental health index may be represented as the number of abnormal markers that have accumulated in a person's neurotransmitter-related biochemistry, expressed in relationship to the number of biomarkers tested and/or an outcome or prognosis of risk of mental illness occurring in future and/or level of severity or disability arising from such mental illness.

Biochemical tests used to determine biomarker levels in accordance with embodiments disclosed herein may be carried out utilising any means known in the art and the present invention is not limited by reference to the means by which the biomarker levels are determined. Determination of biomarker levels may comprise detection and/or quantitation and the methods and techniques available for such determination are well known to those skilled in the art. Suitable methods and techniques include, but are not limited to, the use of spectral analysis, column chromatography, gel electrophoresis, mass spectroscopy and identification of protein spots, enzyme-linked immunosorbent assay (ELISA), Western blot, image acquisition and analysis (such as magnetic resonance imaging (MRI) spectroscopy and single photon emission computed tomography (SPECT)). Biochemical tests used to determine biomarker levels in accordance with embodiments disclosed herein may be employed in any suitable environment or setting, such as a hospital, clinic, surgical or medical practice, or pathology laboratory. Alternatively, or in addition, such biochemical tests may be incorporated into one or more devices capable of analysing the desired biomarkers to thereby allow a degree, or complete, automation of the testing process. Suitable devices are typically capable of receiving a biological sample, analysing one or more biomarker levels in said sample and providing data on said biomarker level(s) in real time thus facilitating bench-to-bedside and point-of-care analysis, diagnosis, risk assessment and/or treatment. Suitable devices include, but are not limited to, the Cobas® in vitro diagnostic systems (Roche Diagnostics). The device may be a handheld device.

In accordance with particular aspects as disclosed herein there are provided methods for predicting the susceptibility of a subject to a mental or neurodegenerative disorder, or diagnosing a mental or neurodegenerative disorder in a subject, comprising: obtaining one or more biological samples from the subject; determining the levels of one or more biomarkers in the sample(s), wherein the biomarkers are selected from pyrroles, histamine, methionine adenosyltransferase (MAT) activity, homocysteine, copper and zinc; and comparing the level(s) of the biomarker(s) determined in (b) with the level(s) of said biomarker(s) from one or more control samples, wherein abnormal levels of the one or more biomarkers in the sample(s) from the subject compared to the one or more control samples is predictive of susceptibility of the subject to a mental or neurodegenerative disorder or diagnostic of a mental or neurodegenerative disorder.

In accordance with another aspect as disclosed herein there is provided a method for diagnosing a mental or neurodegenerative disorder in a subject, the method comprising: obtaining one or more biological samples from the subject; determining the levels of a panel of biomarkers in the sample, the biomarkers including pyrroles, histamine, MAT activity, homocysteine, copper and zinc; and comparing the levels of the biomarkers determined in (b) with the levels of said biomarkers from one or more control samples, wherein abnormal levels of at least three of the biomarkers in the sample from the subject compared to the one or more control samples is indicative of a mental or neurodegenerative disorder in the subject.

As disclosed herein other aspects and embodiments provide methods and assays for evaluating the mental or neurodegenerative disease control in a subject, monitoring disease control in a subject over time, evaluating the efficacy of a treatment or disease management regime, designing an appropriate treatment or disease management regime for an individual, and identifying compounds for use as novel therapeutic agents for the treatment of mental and neurodegenerative disorders.

For the purpose of diagnosing mental and neurodegenerative disorders, diagnoses made in accordance with embodiments disclosed herein, may be correlated with or determined in conjunction with conventional psychiatric diagnoses, for example as generally exemplified by the International Diagnostic and Statistical Manual of Mental Disorders, Fourth Edition, (DSM IV or DSM IV-R) (the disclosure of which is incorporated herein by reference in its entirety) or other international mental disease classification systems known to those skilled in the art. Thus, embodiments disclosed herein find application in the diagnosis, assessment and treatment of a range of mental disorders, including but not limited to, depression, mania, bipolar disorders such as bipolar affective disorder, central auditory processing disorder, somatoform disorder, complex regional pain syndromes, schizophrenia, schizoaffective disorder, schizophreniform disorder, delusional disorder, brief psychotic disorder, oppositional defiance disorder, shared psychotic disorder, Asperger's disorder, Anorexia and/or bulimia (as an expression of Aspergers Disorder), personality disorders, autism, obsessive-compulsive disorder, attention deficit disorder, dyslexia (as an expression of attention deficit disorder), epilepsy, narcolepsy and cataplexy (as expressions of extreme attention deficit disorder), attention deficit hyperactivity disorder, conduct disorder, generalised anxiety disorder, intellectual delay, substance-related disorders, childhood disorders, dementia including Alzheimer's disease, autistic disorder, adjustment disorder, delirium, multi-infarct dementia, suicidality including self harm, suicide-thought, suicide-threat, suicide attempt and completed suicide, psychosis (abnormal, altered state of mind with so-called positive features of sensory disturbance, thought disorder, hallucinations, bizarre behaviours or beliefs (delusions) and/or extreme anxiety and/or negative features in social, cognitive or speech capacity), chronic, acute, delayed, sub-threshold or resolved post traumatic stress disorder (PTSD), schizoptypal personality, schizoid personality and Tourette's disorder, as described in DSM IV or DSM IV-R.

Neurodegenerative disorders to which embodiments disclosed herein may be applied include, but are not limited to Parkinson's disease, motor neurone disease, multiple sclerosis, amyotrophic lateral sclerosis, Alzheimer's disease and other dementia disorders, glaucoma, pseudoexfoliative syndrome and pseudoexfoliative glaucoma.

Moreover, embodiments disclosed herein find application in the diagnosis (and thus in the assessment and treatment) of mental and neurodegenerative disorders by virtue of the symptoms or symptoms groups displayed by subjects. Without limiting the scope of the present disclosure, exemplary symptoms are somatic concern, anxiety, depressed mood, suicidality, guilt, hostility, aggression, elated mood, grandiosity, pressure of speech, suspiciousness/persecution, auditory or visual hallucinations, ideas of reference or control, unusual or bizarre thought content, thought disorder, bizarre behaviour, self neglect, self-harm, threats to others, disorientation, conceptual disorganisation, blunted or flat affect, emotional withdrawal, apathy, social withdrawal, social anxiety, motor retardation, tension, uncooperativeness, excitement, inattention, distractibility, motor hyperactivity, mannerisms or posturing, movement disorder, delusions, poor rapport, passivity, poor abstract thinking, reduced or absent theory of mind, reduced insight, reduced judgement, reduced memory, anti-social traits, tendencies or acts, chronic regional pain or other unexplained chronic pain syndrome, offending behaviour of a forensic nature, disturbance of volition, poor impulse control, anger, delayed gratification difficulty, affective lability, mood lability, mood swings, active social avoidance, preoccupation, obsessional preoccupation, ruminations, disturbance of spontaneity or flow of conversation, poor self care, anxious worrying, tension, tonicity, grasp strength, rumination, fear, active/intentional and passive/unintentional avoidance, dissociation, stress, attenuated psychotic symptoms, overvalued ideation, brief intermittent psychotic symptoms, subjective self-disturbance, re-experiencing phenomena, sense of presence, distancing, corporeality, disturbed stream of consciousness, self-other boundary disturbances, self-demarcation disturbances, body image disturbances, anorexia, orientation and re-orientation disturbances, self-consciousness, first rank passivity symptoms, ideas of reference or control, loss of sense of self, thought insertion, thought broadcasting, thought blocking, thought replacement, abnormal perception, delusional attribution or interpretation, under-arousal, disinhibition, impulsivity, over-aropusal, difficulty attending, reduced attention span, scattered attention, distressing recollections, emotional dysregulation, implausible belief, obsessional compensations, intrusive auditory thoughts, euphoria, apathy, and irritibility.

Methods disclosed herein may be used to predict or determine disorder phenotype, severity and/or disability. For example, abnormal levels of three or more biomarkers may form a pattern of abnormality that demonstrates actual association and has predictive association with other biomarkers to provide a phenotype or prediction of susceptibility to a phenotype of the mental or neurodegenerative disorder that has already been experienced by the subject or may be experienced in the future. Similarly, as exemplified herein, subjects accruing three or more abnormal biomarkers may display clinical global estimation of severity scores significantly greater than that for subjects accruing only one or two abnormal markers, and subjects accruing three or more abnormal biomarkers may display global assessment of function scores significantly less than that for subjects accruing only one or two abnormal markers.

In terms of determining, in accordance with the present disclosure, the efficacy of a treatment regime and determining or designing an efficacious treatment regime for a subject, where for example abnormal biomarkers and phenotype evaluation indicate that a specific neurotransmitter synthesis is likely to be low, a medication aimed at specifically increasing such neurotransmission can be selected with greater confidence of efficacy. Conversely such treatment can be withheld in a situation where biomarker and phenotype evaluation indicate that there is a risk of further increasing neurotransmitters in situations where abnormal biomarkers indicate that neurotransmitter synthesis is likely to be in excess and therefore detrimental to mental state.

Further, by way of example, if a subject with depression is found to have high histamine levels, it is likely that they will have low serotonin synthesis and therefore respond well to a selective serotonin reuptake inhibitor medication. Further, by way of example, if a subject with depression is found to have high histamine levels as a surrogate marker of undermethylation, it is likely that they will have low dopamine synthesis and if they also have attention deficit symptoms, may therefore respond well to a dopamine-enhancing stimulant medication such as methyl-phenidate or Dexamphetamine. Further, by way of example, if a subject with depression is found to have high histamine levels, it is likely that they will have low noradrenalin synthesis and if they also have attention deficit symptoms, may therefore respond well to a noradrenalin-enhancing medication such as Atomoxetine Hydrochloride. Further, by way of example, if a subject with depression is found to have high histamine levels, it is likely that they may have low noradrenalin and serotonin synthesis and if they also have depression symptoms, may therefore respond well to a serotonin-noradrenalin-enhancing medication such as Venlafaxine or Duloxetine. Also by way of example, a subject with depression and sleep disturbance (or other mental state disturbance) and high histamine levels may respond well to an antihistamine medication. Also by way of example, if a subject with psychosis has a high histamine level, it is possible that a conventional dopamine blocking medication will increase their abnormal symptoms, and should be avoided. Conversely, a subject with a low histamine level may not benefit and/or may be adversely affected by treatment with a selective serotonin reuptake inhibitor medication or a dopamine enhancing stimulant medication. An antipsychotic dopamine blocking agent or a beta-adrenergic blocking agent may be indicated for a subject with low histamine levels. Further, a reduction in urinary pyrrole (e.g. HPL) levels and/or an increase in plasma homocysteine levels may be indicative of efficacy of the treatment regime.

Without wishing to be bound by theory, the inventor suggests that because the effect of low histamine and/or elevated urinary pyrrole levels produce a mixed picture of dopamine neurotransmitter excess, serotonin depletion and noradrenergic excess from high copper load, low histamine and elevated urinary pyrrole levels may be involved in the aetiology of mixed affective states such as bipolar affective disorder. For this reason it is expected that either or both low histamine and elevated urinary pyrroles will be associated with a positive response to gamma amino butyric acid agonist agents (load such as sodium valproate), which dampen the over excitability cause by overmethylation and high copper load. This also predicts a positive response to lithium (which acts to dampen excessive dopamine signalling through a number of gene regulation pathways as well a having a direct regulatory effect on tyrosine hydroxylase and NMDA receptor inhibition).

In accordance with embodiments disclosed herein, histamine is typically a surrogate marker of methylation activity, in particular the activity of methionine adenosyltransferase (MAT), such that elevated histamine levels are indicative of reduced MAT activity (undermethylation). MAT catalyses transfer of the adenosyl group of ATP to the sulfur atom of methionine. A major path of metabolism of serum histamine is by its acceptance of a methyl group from S-adenosyl-methionine (SAMe OR AdoMet). SAMe levels are in turn influenced by the activity of MAT.

MAT has a variety of synonyms, including S-adenosylmethionine synthetase, adenosyl methyltransferase, AdoMet synthetase 1, ETH10, L9470.9, MAT 1; methionine adenosyltransferase 1, S-adenosylmethionine synthetase, S-adenosylmethionine synthetase 1, YLR180W, adenosylmethionine synthetase, ATP-methionine adenosyltransferase, ATP:L-methionine S-adenosyltransferase, methionine S-adenosyltransferase, methionine-activating enzyme, S-adenosyl-L-methionine synthetase, and S-adenosylmethionine synthase. The scope of the present disclosure encompasses all MAT synonyms as well as MAT variants, homologues and isoenzymes. MAT isoenzymes include, for example, MAT I, MAT II and MAT III.

MAT activity may be detected and measured indirectly (for example via serum histamine levels as exemplified herein) or directly. Direct measurement of MAT levels and/or activity may be made in any bodily tissue or fluid, including for example, erythrocytes and brain samples. Determination of MAT activity may comprise, for example, analysing kinetic parameters of MAT activity including maximal enzyme velocity (Vₘₐₓ), and substrate affinity (for example the Michaelis constant, K_{M}). In accordance with embodiments disclosed herein, MAT measurements may also comprise measurements of the activity of MAT catalytic subunits and analysis of gene expression (of, for example, MAT1A, MAT2A and MAT2B).

A biological sample for use in accordance with embodiments disclosed herein may comprise one or more fluid or tissue samples. Samples may comprise blood, urine, sputum, saliva, stool, lysed cells, breath, condensed breath, cerebrospinal fluid, amniotic fluid, any other body fluid, and tissue sections. The sample may comprise fresh, frozen or otherwise stored biological material. In some circumstances, the sample may undergo treatment, incubation or culturing after extraction from the subject. Tissue samples may be derived from postmortem or from live subjects. Typically, biological samples employed in accordance with the invention include blood and urine. The blood may comprise erythrocytes, leukocytes, whole blood, serum, or more typically plasma.

As detailed hereinbefore, the development of the present invention is based on the determination that the levels of a panel of biomarker levels in patients suffering from a mental or neurodegenerative disorder are dysregulated relative to normal endogenous levels of such markers. Accordingly, reference to “normal endogenous levels” should be understood as a reference to the levels of the biomarkers in one or more subjects that does not suffer from, and/or is assessed to have no predisposition to, a mental or neurodegenerative disorder. It would be appreciated by the person of skill in the art that this “normal level” is likely to correspond to a range of levels, as opposed to a singularly uniform discrete level, due to differences between cohorts of individuals. By “cohort” is meant a cohort characterised by one or more features which are also characteristic of the subject who is undergoing treatment. These features include, but are not limited to, age, gender or ethnicity, for example. Accordingly, reference herein to elevated or reduced biomarker levels relative to normal endogenous levels is a reference to increased or decreased biomarker levels relative to either a discrete level which may have been determined for normal individuals who are representative of the same cohort as the individual being treated, or relative to a defined range which corresponds to that expressed by a population of individuals corresponding to those from a range of different cohorts. Similarly, those skilled in the art will appreciate that the term “abnormal” refers to levels of biomarkers that fall outside the range of normal endogenous levels determined for a particular biomarker.

According to embodiments disclosed herein the levels of pyrroles, histamine, MAT activity, homocysteine, free copper, zinc and ratios of zinc to copper can be used alone or in combination as biomarkers for the diagnosis of mental and neurodegenerative disorders and for the control or status of the disorder in a subject. Those skilled in the art will readily appreciate that the absolute value of the biomarker levels may vary depending on circumstances and the invention is not limited by the specific values exemplified herein. Rather, in practicing assays and methods in accordance with embodiments disclosed herein the biomarker levels determined for any particular subject will typically be compared to one or more control levels used as a reference in order to achieve the desired diagnosis.

Thus, the term “control” or “control sample” as used herein refers to one or more biological samples from individuals or groups of individuals classified as not having the particular mental or neurodegenerative disorder(s) for which a subject is being assessed and where the diagnosis for the “control” or “control sample” has been confirmed. A “control sample” may comprise the compilation of data from one or more individuals whose diagnosis as a “control” for the purposes of the present invention has been confirmed. That is, for the purposes of practicing embodiments of the present invention samples to be used as controls need not be specifically or immediately obtained for the purpose of comparison with the sample(s) obtained from the subject under assessment.

It will be appreciated that methods disclosed herein may be used in conjunction with one or more alternative diagnostic and assessment methods for mental and neurodegenerative disorders known to those skilled in the art.

Embodiments disclosed herein also contemplate the use of one or more additional biomarkers to aid in the diagnosis, prediction, or other assessment of a mental or neurodegenerative disorder. Such additional biomarkers may, for example, be used to validate or extend diagnoses, predictions or assessments made in accordance with the present disclosure, further predict or assess disability or severity, or predict or assess whether the subject tested suffers from any other underlying disorder that may further impact on mental or neurodegenerative disorder diagnosis. Such additional markers may be markers of, for example, inflammation, tissue damage, urine excretion function and histamine metabolism. Suitable ‘validation’ markers may include, for example, 1-methyl histamine, histidine, S-adenosyl-methionine, S-adenosyl homocysteine, ratios between S-adenosyl-methionine and S-adenosyl homocysteine, serum/plasma adenosine, reduced and oxidised glutathione and ratios between these two forms of glutathione, red cell pyroxidine activation test, 25-hydroxy 2 D3 (calcitrol, vitamin D), red cell fatty acids with AA (arachadonic acid)/EPA (eicosapentanoic acid) and DHA (docosahexanoic acid) estimation, urine or plasma tetrohydrobiopterin, 5-hydroxy indole acetic acid, platelet monoamine oxidase, red cell N-methyl transferase, catechol-O-methyltransferase polymorphisms, methyl tetrahydrofolate reductase polymorphisms, urine homovallinate, vanilmandelic acid, serum creatinine, immunoglobulins A, E, G, & I, IgG and IgE food allergy screens, IGE allergy correlates, inflammatory cytokine levels, C reactive protein, serum iron (ferritin, transferrin, transferrin saturation), serum, plasma or urinary lead, antigliadin antibodies, red cell folic acid, serum vitamin B12, red cell/serum magnesium, vitamin B12, serum calcium, free calcium concentration, blood sugar and plasma insulin, N-acetylaspartate, D glucaric acid, phosphocreatinine, glutamate dehydrogenase, N methionine adenosyl transferase, plasma retinol, B-retinylacetate, B-retinoic acid, bexoratene, tyrosine hydroxylase, thyroxine T3, T4 and reverse T3 components, serum creatinine, prostoglandin E1.

Further additional markers that may be measured or assessed in conjunction with the biomarkers hereinbefore disclosed and in accordance with embodiments disclosed herein include, but are not limited to: urinary porphyrins including total urinary haeme, urinary precoproporphyrin (COPRO), keto-isococoporphyrin, urinary uroporphyrin (URO), urinary precoproporphyrin (PRECOPRO), PRECOPRO:URO ratio, uroporphyrin decarboxylase (UROD), cocoporphyrinogen oxidase (CPDX), hepta and hexacarboxyporphyrins, 5-aminolevulinic acid (gamma ALA), urinary coproporphyrinogen and faecal isococproporphyrin); serum/plasma 1 methyl histamine; tGSH:GSSG ratio; glutathione peroxidase; superoxide dismutase; glutathione S transferase P1 (GST P1); glutathion-S-transferase M1 (GST M1); urinary alphahydroxybutyrate; urinary DHPG:MHPG ratio; urinary pyroglutamate; urinary sulphate; urinary 8-hydroxy-2-deoxyguanosine; red cell folic acid; red cell methyl malonic acid; urinary forminoglutamate; methyl tetrahydrofolate reductase polymorphisms (MTHFR); serum/plasma adenosine; red cell pyridoxine activation test; red cell transketolase; red cell pyrodoxal phosphate activation test; plasma cysteine; total glutathione (reduced) glutathione (GSSG); urinary or plasma tetrahydrobiopterin BH4; red cell pyridoxine activation test; red cell transketolase; urinary xanthurenate; urinary kyenurate; 25 hydroxy cholecalciferol; vitamin D receptor polymorphisms; urinary DOPAC:HVA ratio; vitamins CoQ10, E, A, or D; urinary adipate; urinary suberate; urinary ethylmalonate; APOE polymorphisms; urinary methylmalonate; serum/plasma methionine; serum/plasma S adenosyl methionine; red cell magnesium; serum magnesium; serum Fe 10; ferritin; transferrin; serum cortisol; DHEAS; urine imidazole acetic acid, whole blood histamine, substance P; urinary alpha-ketoisovalerate; urinary alpha-ketoisocaproate; urinary alpha-keto-b-methylvalerate; urinary beta-hydroxyisovalerate; urinary HIAA (5-hydroxyindoleacetic acid); urinary DOPAC (3-methoxytyramine); histidine methyl transferase, urinary HVA (homovallinate); urinary DHPG (dihydroxyphenylglycol); urinary MHPG (urinary 3-methoxy-4-hydroxyphenylglycol); urinary DOMA:VMA; red cell catechol-o-methyl transferase (COMT) including polymorphisms; mRNA for 7 nicotinic acetylcholine receptor, choline creatinine ratio, phosphocreatinine, alpha C-methyl-L-tryptophan trapping, N acetyl aspartate, eosinophil protein X and eosinophil calprolectin, plasma S adenosyl homocysteine; S adenosyl homocysteine hydrolase; methyl tetrahydrofolate reductase polymorphism (MTHFR); platelet catecholamines; urinary hydroxymethylglutarate; blood lymphocyte 7 nicotinic acetylcholine receptor, IGG food allergy screen; imidazole N-methyl transferase, B2 microglobulin; antigliaden autoantibodies (such as tissue transglutamase IGG, tissue transglutaminase IGA, Methionine adenyl transferase, endomysial antibody); urinary p-hydroxyphenylacetate; CD8 and SD4 T cell levels; inflammatory cytokine levels; urine methyl histamine, urine histamine, C reactive protein; erythrocyte or serum N methyl transferase, nerve growth factor; arginine N methyltransferase; urinary VMA (vanilmandelic acid); vesicular monoamine trasnporter (VMAT2); neuronal nitric oxide synthetase; alpha-C methyl-L-tryptophan; acetyl cholinesterase, choline acetyltransferase; vesicular acetylcholine transporter; and tyrosine hydroxylase; red blood cell choline, alpha 7Acetylcholine receptor activity, alpha 4 acetylcholine receptor activity, acetylcholine esterase, glutamic acid decarboxylase, taurine, adenosine, kainate, glycine, spermine, spermidine, glutamate, substance P, aspartate, biotin, quinolones, quinolinate, quinolinic acid, picolinate, kyeneuric acid, free androgen index, urinary phydroxyphenylacetate, serum histidine, urinary DOMA (3,4-dihydroxymandelic acid); plasma nitrous oxide; Cu:Zn ratio (N 0.8-1.2); free copper (Cu); urine histamine; plasma chromium; whole blood serum and urine lead (Pb), mercury (Hg) and cadmium (Cd); hair mineral analysis for cadmium, mercury, arsenic, lead, copper, chromium, lithium, sodium, potassium, bismuth and chloride; urine.whole blood, red cell and/or serum assays of vitamin D, 250H vitamin D, vitamin D receptor (VDR), vitamin A, B6, serum B12, plasma methyl-malonic acid; plasma, blood and/or urine assay of pyridoxine-5-phosphate (P5P), pyridoxil kinase, niacin, niacinamide, red cell transketolase; thyroid stimulating hormone; thyroid peroxidase antibodies; free T3 and T4; reverse T3; serum cortisol; urinary iodine, urinary folate as urinary fromino-glutamic acid (FIGLU); urinary N-methyl-Nicotinamide.methylmalonic acid; erythrocyte glutamic-pyruvic transaminase (EPGPT); glutamic-oxaloacetic transaminase (EGOT); serum levels of electrolytes, creatinine and Ca⁺⁺, Mg⁺⁺ and BSL; ferritin; biopterin; C— reactive protein (CRP); serum and/or red blood cell assay of manganese; secretory IGA; serum IGA, IGG, IGM and IGE; IGG and IGE for gluten and casein sensitivity; red cell fatty acids; arachadonic acid (AA):EPA ratio; lipid peroxidises; H₂O₂; t-butylhydroperoxide; cumene hydroperoxide; 2-thiobarbituric acid reactive substances (TBARS); apometallathionein; glutathione disulphide (GSSH); total glutathione; reduced glutathione (GSH); GSH:GSSH ratio; glutamic decarboxylase; oxidative stress biomarkers including 8 hydroxydeoxyguanosine (8-OhdG), malondialdehyde (MDA) and isoprostane; glutathuione peroxidase (GSH-Px); superoxide dismutase (SOD); urine lipid peroxides; hydroxy catechol markers; glutathione transferase; S adenosylhomocysteine hydrolase; spinal motor neuron survival gene (SMN); red cell and/or serum methionine adenosyltransferase; S-adenosyl-L-methionine synthetase; methionine breath test; adenosine deaminase; urinary indicans; valerate isobutyrate; urine analysis of lactulose, mannitol and lactulose:manitol ratio after lactulose mannitol challenge; serum cholesterol; triglycerides; uric acid; serum iron; ferritin, transferrin and transferrin saturation; aspartate amino transferase (ALT), alanine amino transferase (AST); lactic dehydrogenase (LDH); low density lipoprotein (LDL); tissue transglutaminase IgG; tissue transaminase IgA; endomysial antibody; calprotectin and eosinophil protein X; interleukin IB; serum testosterone; free androgen index; DHEAS (dehydroepiandosterone); antigliadin IgA; serum transglutaminase IgA antibody; gliadin IgG antibody; full blood count; haemoglobin; faecal PH; cholesterol; pancreatic elastase; n butyrate; acetate; propionate; faecal total short chain fatty acids; total long chain fatty acids; faecal microbiology, mycology and parasitology; glycine:glucuronide ratio; sulphate:glucuronide ratio; sulphate:creatinine ratio; cytochrome P450 genotype; D glucaric acid; glutamate dehydrogenase; C/Zn superoxidedismutase; urinary amino acids: histidine, isoleucine, leucine, lysine, methionine, phenylalanine, threonine, tryptophan, valine, cysteine, glutamine, taurine, tyrosine, alanine, arginine, aspartic acid, glutamic acid, glycine, proline, serine, aspartate, asparagine, tyrosine, glutamine and glutamate; copper/zinc superdioxide and catalase activity; ESR; IL-1B (interleukin 1B); tumour necrosis factor alpha (TNFalpha) and serum alpha1, alpha2 and gamma fractions; platelet glutamate levels; serum holotranscobalamin; NMDA receptor NR2B subunits and other sub unit receptor activity; blood trype; prostoglandin E1; brain derived neurotrophic factor Val/Met polymorphisms; 5HTT-LPR polymorphisms; thiamine; omega 3; omega 6; retinoic acid; bexoratene; UA B30; blood diamine oxidase activity; blood or urine urea; creatinine levels; blood or serum thyroxine T3, reverse T3 and T4 components; urine ammonia concentration; urinary amino-n-butyric acid; foramino glutamic acid; urine anserine; urine sarcosine; alpha-ketocaproic acid; beta aminoisobutyric acid.urine glutamic acid; glutaric acid; and glutamine/glutamate ratio; pyroglutamic acid; 3-hydroxypropionic acid. dihydroxyphenylpropionic acid; urine arginine/ornithine ratio; citruline; kyneuric acid; serine; tyrosine; 3 methoxy-4-oh-phenylglycol; threonine; taurine; 4-hydroxyphenylpyruvic acid; suberic acid; pyruvic acid; 5 hydroxyphenylpyruvic acid; citric acid; cisaconitic acid; citric acid; aspartic acid; lactic acid; adipic acid; phenyl acetic acid; 5-hydroxy indolacetic acid; dihydroxyphenylpropionic acid; 2-hydroxyphenylacetic acid; cysteine homogentisic acid; benzoic/hippuric acid ratio; lipid peroxidases; carnosine; alpha-amino-n-butyric acid; alpha ketovaleric acid; alphaketomethylvaleric acid; alpha ketovaleric acid; succinic acid; urine beta aminoisobutyric acid; gamma-aminoisobutyric acid; indoleacetic acid; phenylacetic acid; arabinose; malic acid; homogentistic acid; urine methylmalonic acid; urine homocysteine; urine 1-methyl histidine; 3-methyl histidine. urine succinyl purine; inosine; adenosylcobalamine coenzyme; proline; phosphoserine; ethanolamine; urine phosphoserine; urine cystathione; phosphoethanolamine; orotic acid; urine n methylglycine; urine opiate peptides; IgG and IgE anti casein and gluten antibodies; plasma serine; plasma glycine; serine hydroxymethyltransferase; C¹⁴ or C¹¹ labelled CO₂ following C¹⁴- or C¹¹-methionine administration; histamine N methyl transferase (HMT); serum/plasma glycine; methylcytosine binding protein (MeCP2); histone(H4) deacetylase; acetylated histone(H4); plasma pyridoxyl phosphate; glutathione-S-transferase; cystathione beta synthetase (CBS, CbetaS); plasma alkalinepyridoxine phosphate phosphatise; mitogen phytohemagglutin (PHA); serum histamine(2-(4-Imidazolyl)-ethylamine); red blood cell histamine; erythrocyte histamine-N-methyltransferase; glycine-N-methyltransferase; retinol binding globulin; glutathione-S-transferase; urine dimethyltryptamine (DMT); fasting blood alanine; blood lactate:pyruvate ratio; blood acetyl-carnitine: free carnitine ratio; beta casomorphin-7; casomorphin; influenza titre; glutamic acid decarboxylase 65 & 67 KDA; Reelin proteins; plasma rennin; serum creatinine; serine hydroxymethyltransferase; glutathione synthetase; heart rate; blood pressure; continuous task performance; saliva cortisol; catecholamines (noradrenalin and adrenalin and metabolites); corticotrophin releasing factor; and coeliac screen. For biomarkers listed above, measurements may be made of levels, ratios and/or activities, as appropriate.

In diagnosing and assessing mental and neurodegenerative disorders in accordance with embodiments disclosed herein, determination of biomarker levels as disclosed herein may be used in conjunction with a range of other tests known and available to those skilled in the art including, for example, psychiatric and behavioural tests. For example, in the case of central auditory processing disorder, additional tests and analyses that may be employed include: an acoustic reflex and reflex decay test; anxiety potentiated startle reflex; startle reaction time; acoustic startle (threshold, inhibition and affective inhibition); auditory (and visual) processing tests such as auditory (and visual speed) of processing test, auditory (and visual) working memory tests, auditory tone (pitch) discrimination test, division of auditory attention test, filtered word test, auditory figure ground test, competing words test, (3) MeV, visual field evoked response test, and prepulse inhibition test; auditory brain stem responses (ABR) such as stimulus threshold, waveform morphologies, absolute and relative amplitudes, latencies, middle latency response (MLR) and relative interpeak latencies for ABR waves N1, Na, Pa, Pb and late latency response (LLR), N1, P2 and P3 (P300) components; quantitative EEG and topographic mapping of alpha, beta, theta and delta waves and all possible power ratios between these waves, including absolute power, relative power and power relative to normal data base, spectral analysis, independent component analysis, Z score analysis and signal source analysis; visual response search score; auditory and visual processing speed; eye blink rate; mismatch negativity; retrograde memory; immediate memory; memory selection; executive function; N-back test; response speed; directed ignoring task; go/nogo response inhibition; internal/external locus of control; strength of memory score; Neale analysis reading test; memory tests (e.g. Ray copy/recall, RAVLT and RAVLT errors, SILS, quick T, IT); saccadic eye movements; antisaccade task; verbal fluency and FAS test; EEG gamma band synchrony; and auditory (and visual) evoked response tests, components including mismatch negativity component (MMN), N1, P50, P400, P3 and P3b components during a cognitive task, contingent negative variation component (CNV) and post-imperative negative variation (PINV) component.

Embodiments disclosed herein provide methods for evaluating disease control in a subject, monitoring disease control in a subject over time, evaluating the efficacy of a treatment or disease management regime and designing an appropriate treatment or disease management regime for an individual. For example, methods may comprise determining whether the levels of selected biomarkers are within a predetermined range indicative of satisfactory control or management of the mental disorder. A level outside of a predetermined range may indicate that the subject's disease treatment or management needs to be modified to improve disease control or that the subject should otherwise be placed on a suitable treatment regime. The analysis may be repeated one or more times over a given period of time to monitor disease control in the subject over time. Determination of the disease control in a subject, in particular the monitoring of control over time, also facilitates decision making with respect to the most appropriate intervention or treatment regime for an individual subject. The treatment regime will exemplify personalised medicine practices in that it will typically be tailored so as to obtain a normalisation in the levels of selected biomarkers. For example, this may comprise introducing a new treatment regime or modifying an existing regime with a view to improving disease symptoms or other parameters. The modification of a regime may be modification with respect to any one or more of a variety of factors, such as the nature of any existing medication, the dosage thereof, the timing of administration and/or any supplementary disease management strategies. Such decision making with respect to treatment regimes will vary from case to case and the determination of the most appropriate strategy is well within the expertise and experience of those skilled in the art.

A treatment regime for the treatment of a mental or neurodegenerative disorder in a subject in accordance with an embodiment disclosed herein may involve administration of any medications commonly utilised in the treatment of the particular mental or neurodegenerative disorder in question and/or may involve a variety of other physical medical, psychological and/or psychiatric treatments. In the case of drug administration, the treatment regime may comprise the administration of a number of drugs simultaneously, sequentially, or in combination with each other. The type of drug(s) administered, dosage, and the frequency of administration can be determined by those directing the administration of the drugs in accordance with accepted medical principles, and will typically depend on factors such as the severity of the disease, the age and weight of the subject, the medical history of the subject, other medication being taken by the subject, existing ailments and any other health related factors normally considered when determining treatments.

All essential components required for detecting biomarkers as disclosed herein in samples, typically blood and urine samples, in accordance with methods of the present invention may be assembled together in a kit. The kits may optionally include appropriate components for quantifying biomarker levels, appropriate positive and negative controls, dilution buffers, reagents and the like. Such reagents, buffers, controls and the like may be standardised so as to be suitable for use on any one of a number of devices known to those skilled in the art for the analysis of blood and urine samples. Kits may also comprise devices and/or software to facilitate the employment of methods disclosed herein, for example including suitable computer software to determine or calculate a risk, susceptibility or prognosis based on determined biomarker levels. Typically, the kits comprise instructions for performing the methods of the present invention, optionally together with educative information and an algorithm (decision tree) for treatment recommendations based on the results obtained using methods and kits of the present invention.

All publications mentioned in this specification are herein incorporated by reference. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

It will be appreciated by persons skilled in the art that numerous variations and/or modifications may be made to the invention without departing from the spirit or scope of the invention as broadly described. The present embodiments are, therefore, to be considered in all respects as illustrative and not restrictive.

The present invention will now be described with reference to the following specific examples, which should not be construed as in any way limiting the scope of the invention.

### EXAMPLES

### Example 1

### Subject Recruitment

This study utilised resources at hand in a moderate sized psychiatric clinic with private funding. The study was conducted between January 2007 and May 2008.

Symptomatic participants (the “patients”), consisted of 87 General Practitioner or Paediatrician—referred participants and another recruited 15 asymptomatic, consenting, participants (the “controls”). Participants were aged 2 to 61 yrs, with mixture of patients between child, adolescent & adult population. Patients and controls were free of nutritional supplements and antihistamines and none were on medications known to alter the levels of biomarkers such as serum histamine. Patient pharmacotherapy remained stable over the assessment period.

Informed, consenting patients and controls were DSM-IV-R diagnosed and rated for mental illness symptoms, illness severity and disability. Patients and controls were then referred to a laboratory for blood and urine collection for relevant biochemical markers. Participant rating predated the obtaining of laboratory results by one month.

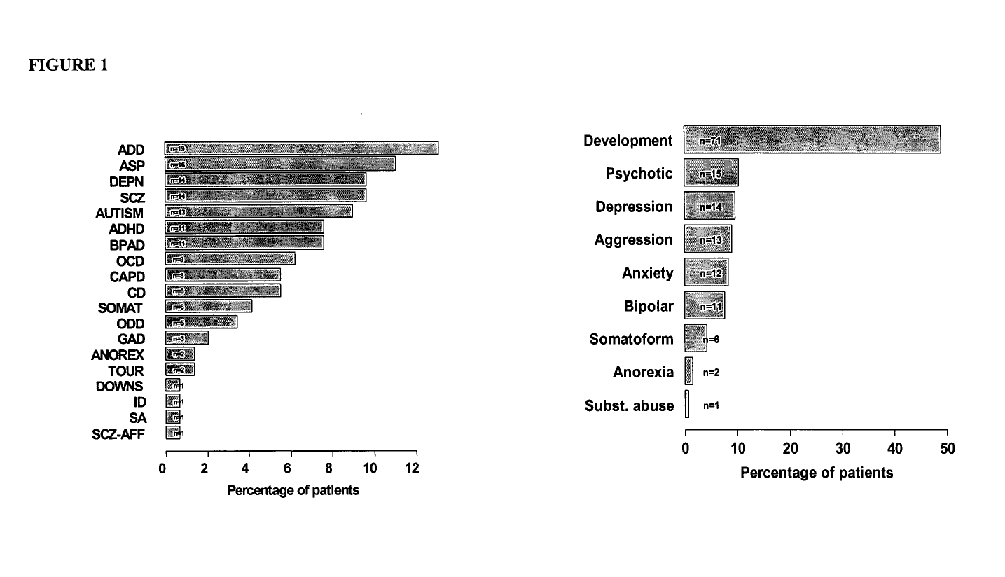

Distribution of diagnostic groups (FIG. 1A) demonstrated a larger number of diagnoses of attention deficit, major depression, schizophrenia and pervasive developmental disorders. Diagnoses were coded as follows: Central Auditory Processing Disorder (CAPD), Depression (DEPN), Somatoform Disorder (SOMAT), Schizophrenia (SCZ), Oppositional Defiance Disorder (ODD), (AUTISM), Attention Deficit Disorder (ADD), Obsessive Compulsive Disorder (OCD), Aspergers Disorder (ASP), Bipolar Affective Disorder (BPAD), Conduct Disorder (CD), Attention Deficit Hyperactivity Disorder (ADHD), Generalised Anxiety Disorder (GAD), Intellectual Delay (ID), Anorexia (ANOREX), Tourette Syndrome (TOUR), Downs Syndrome (DOWNS), Schizoaffective Disorder (SCZ-AFF). Distribution of major symptom groups: anxiety, depression, bipolar, development, somatoform, psychotic, aggression, is demonstrated in FIG. 1B.

### Example 2

### Analysis of Biomarkers in Blood and Urine Samples of Subjects

Using the subjects recruited, and as described, in Example 1 serum and urine levels of a series of biochemical compounds and molecules (markers) were investigated to evaluate their predictive and diagnostic potential for mental disorders. The biochemical markers analysed were urinary HPL, serum histamine, serum homocysteine, free serum copper and serum zinc.

Demographic data was examined, with respect to age, gender, diagnostic groups and symptom groups, then the significance of the distribution and number of abnormal markers accrued by patients versus controls was analysed by the Kolmogorov-Smirnov distribution comparison test (Conover, 1999) and Fisher's exact test for counts (Agresti, 2002). Fisher's exact test of counts was used to determine significant differences between numbers of patients and controls for the biochemical markers in question.

Demographic data for the subjects examined in this study are shown below in Table 1.

TABLE 1

| | Patients | Controls |
|---|---|---|
| Median age | 16 | 50 |
| Average age | 18.5 | 45.8 |
| Gender M/F | 42/45 | 4/11 |

Normal ranges for each of the biochemical markers analysed are shown in Table 2. As per recommendations (Pfeiffer et al, 1975), the total laboratory range of histamine (0.2 to 1.0 umol/L) has been narrowed compared with commercial laboratory reference levels, in order to reflect the two states of high and low methionine adenosyltransferase (MAT) activity i.e. weak action of MAT=high histamine>0.59 umol/L, and over-strong action of MAT=low histamine<0.35 umol/L).

TABLE 2

| Biochemical Test | Normal Range | |
|---|---|---|
| HPL | <15 | ug/dL |
| Histamine | 0.35-0.59 | umol/L |
| Free Cu | 10-25% | |
| Zn | 10-18 | umol/L |
| Homocysteine | 6-14 | umol/L |

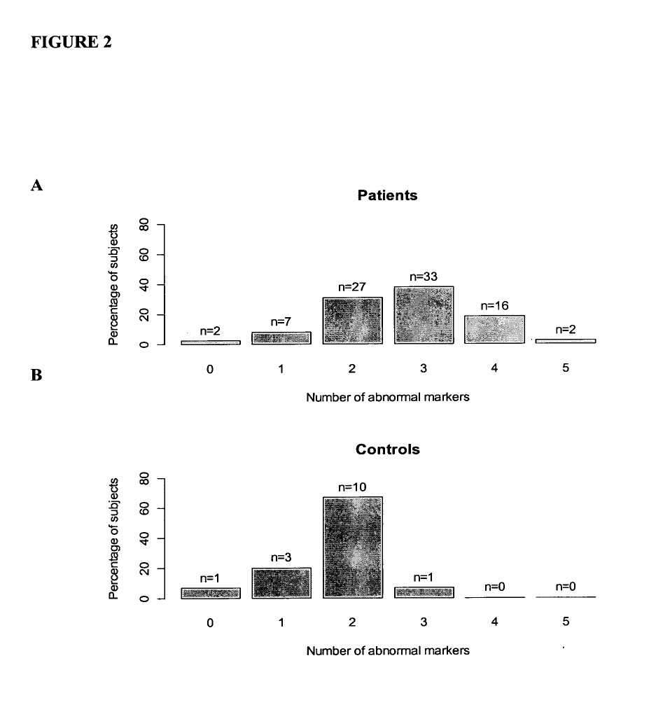

The number and distribution of abnormal markers amongst patients and controls are shown in FIG. 2. Using patients with 0-2 abnormal markers versus 3-5 abnormal markers, the number of abnormal markers was significant for severity (CGI, P=0.005) & disability (GAF, P=0.01).

The distribution of biochemical markers amongst patients was significantly different than for asymptomatic controls using the Kolnmogorov-Smirnov test (Conover, 1999), P=0.002, and on Fishers exact test (Agresti, 2002). Participants who accumulated three or more abnormal biochemical markers (n=51) overall, had a much greater probability (P=<0.001) of attracting a psychiatric diagnosis (see FIG. 2). Indeed, diagnoses of significance that accrued three or more abnormal markers in this group were: Central Auditory Processing Disorder (n=7/8=87% of the patients with CAPD, P=0.0002, compared with 1/15 controls); Somatoform Disorder (n=⅚=83% of patients with that diagnosis, P=0.002); Depression (n= 11/14=79% of all cases of depression, P=0.0001); Schizophrenia (n= 10/15=67% of all schizophrenia cases, P=0.002); Oppositional Defiance Disorder (n=⅘=80% of all cases, p=0.005); Autism (n= 7/12=67% of all cases P=0.008); and Attention Deficit Disorder (n= 10/19=53%, P=0.008).

Other less-significant, diagnostic contributions to the group of patients who accrued three or more abnormal markers were: Obsessive Compulsive Disorder (n=5/9=56%, P=0.015); Aspergers Disorder (n=8/16=50% of all cases, P=0.016); Bipolar Affective Disorder (n=6/11=55% of all cases, P=0.021); and Conduct Disorder (n=4/8=50% of all cases, P=0.032). Interestingly, 64% of cases of Attention Deficit Hyperactivity Disorder (n=7/11, P=0.12) fell in the range of accruing only one or two abnormal markers.

Though there were significant differences in ratings for severity and disability in general between control and patient groups (P=0.001 for all indices), when patients were separated into two groups containing 0-2 and 3-5 abnormal markers, the difference in their ratings for severity & disability was significant for Clinical Global Impression of Severity (P=0.01) and Global Assessment of Function (P=0.005). Also on correlation matrix analysis (Table 3), a high HPL level was found to significantly positively correlate with all measurement indices associated with disease severity and disability. Another finding was that abnormally high copper levels significantly correlated with indices for severity.

TABLE 3

| | MOF | GAF | MBPRS | CGISev | HPL | Hist | HCY | Cu | Zn |
|---|---|---|---|---|---|---|---|---|---|
| MOF | 1 | 0.00 | 0.00 | 0.00 | 0.00 | 0.68 | 0.60 | 0.50 | 0.84 |
| GAF | −0.85 | 1 | 0.00 | 0.00 | 0.02 | 0.27 | 0.11 | 0.35 | 0.94 |
| MBPRS | 0.77 | −0.78 | 1 | 0.00 | 0.00 | 0.80 | 0.61 | 0.05 | 0.41 |
| CGISev | 0.71 | −0.74 | 0.67 | 1 | 0.01 | 0.91 | 0.82 | 0.23 | 0.59 |
| HPL | 0.32 | −0.23 | 0.41 | 0.24 | 1 | 0.67 | 0.14 | 0.61 | 0.58 |
| Hist | 0.04 | −0.11 | 0.03 | −0.01 | −0.04 | 1 | 0.14 | 0.31 | 0.65 |
| HCY | −0.05 | 0.16 | −0.05 | 0.02 | 0.15 | −0.15 | 1 | 0.98 | 0.88 |
| Cu | 0.07 | −0.09 | 0.19 | 0.12 | −0.05 | −0.1 | 0 | 1 | 0.12 |
| Zn | −0.02 | −0.01 | −0.08 | −0.05 | −0.06 | −0.05 | −0.01 | −0.16 | 1 |

MOF, Measure of Function; GAF, Global Assessment of Function; MBPRS, Modified Brief Psychiatric Rating Scale; CGISev, severity of Clinical Global Impression; HPL, urinary HPL; Hist, serum histamine; HCY, serum homocysteine; Cu, free serum copper; Zn, serum zinc.

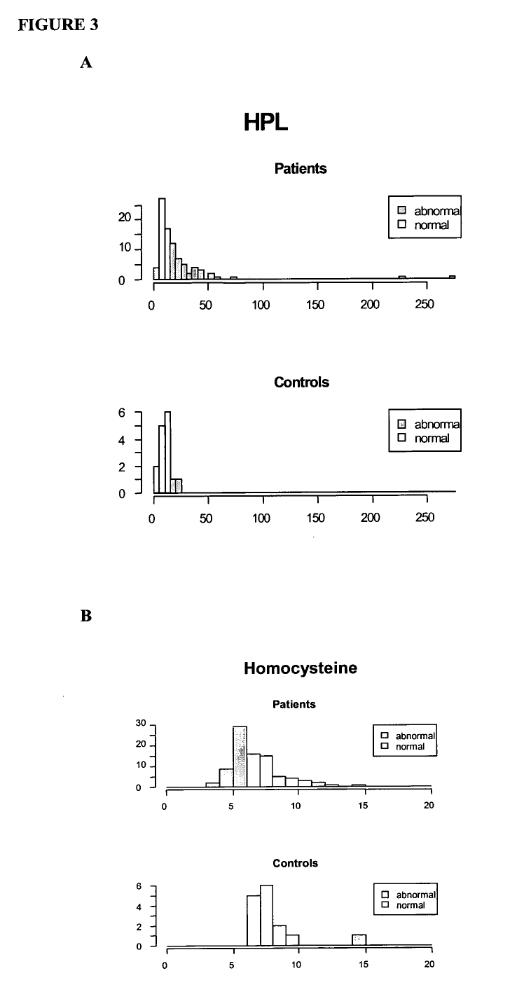

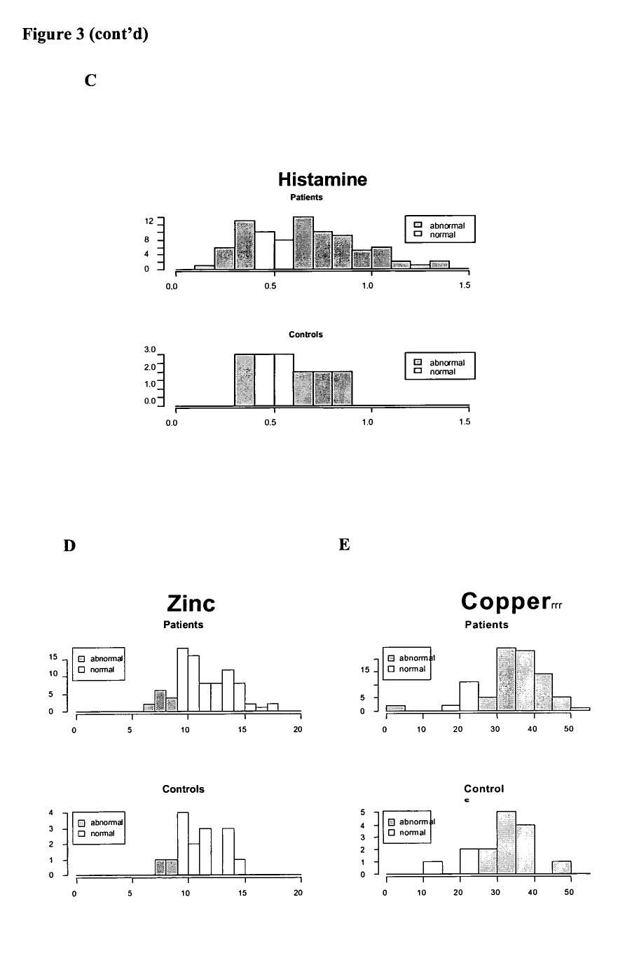

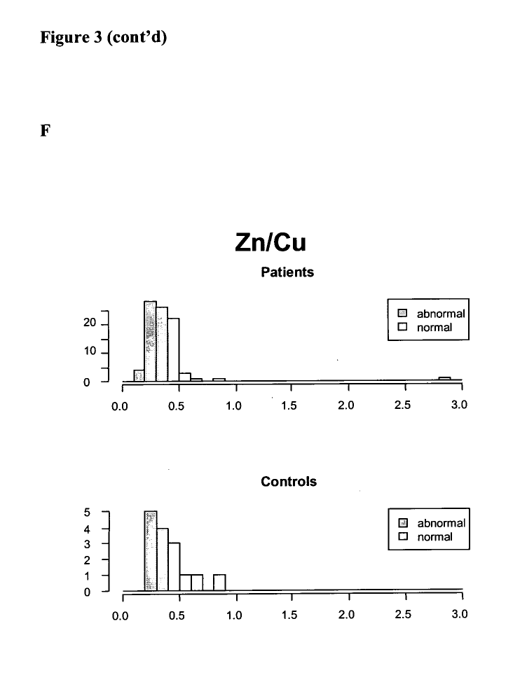

The distribution of levels of HPL, histamine, copper and homocysteine amongst patients and controls was investigated further as shown in FIG. 3.

Using Fisher's exact test to determine significant differences between patients and controls for the biochemical markers in question, there were significantly more patients (P=0.023) with abnormal HPL levels (n=39), than controls (n=2) (FIG. 3A). Overall, 45% of patients (n=39) versus 13% of controls (n=2), had elevated urinary HPL levels. On Fishers exact test for counts, these levels were elevated in 73% of schizophrenia-diagnosed patients (n=14, P=0.023) and 55% of bipolar patients (n=6/11, P=0.08). Also, on Poisson regression, elevated urinary HPL levels had a predictive association of P=0.065 for psychotic symptoms. When elevated pyrroles was present together with low homocysteine, there was a significant predictive association with developmental delay disorders (P=0.01). Elevated HPL levels also correlated significantly with all severity and disability measurement scores on data-correlation matrix.

There was also a significantly (P<0.001) greater proportion of abnormally low homocysteine levels in patients (n=64/87) compared with controls (n=1/15) (FIG. 3B). Patients with low homocysteine of significance had diagnoses of: Central Auditory Processing Disorder (n=⅝, P=0.009, compared to 1/15 controls); Attention Deficit Disorder (n= 10/19, P=0.008); Oppositional Defiance Disorder (n=4/5, P=0.005); Autism (n= 7/13, P=0.011); Aspergers Disorder (n= 8/16, P=0.016); Depression (n= 6/14, P=0.035). The following diagnoses were of less significance: Somatoform Disorder (n= 3/6, P=0.053); CD Conduct Disorder (n=⅘, P=0.10); BPAD Bipolar Affective Disorder (n= 4/11, P=0.13); ADHD Attention Deficit Hyperactivity Disorder (n= 4/11, P=0.13); SCZ Schizophrenia n= 5/15, P=0.17) and OCD Obsessive Compulsive Disorder (n= 4/9, P=0.47). On Poisson regression, anxiety ratings were at their highest level when homocysteine level was lowest.

When low homocysteine was examined in relationship to diagnoses segregated according to their predominant symptom group, all symptom groups were found to have a significantly greater proportion of depressed homocysteine levels compared to the controls. Ranked according to degree of results-significance, these were developmental delay (n=29/57, (51%), P=0.0004), aggression (n=7/13 (54%), P=0.002), anxiety (n=6/12 (50%), P=0.004), depressed mood (n=6/14 (43%), P=0.02), mood instability (n=4/11 (36%), P=0.03), psychotic (n=5/15 (33%), P=0.04). On analysis of symptom-intensity scores from the MBPRS, depressed levels of homocysteine had significantly lower scores for depression and withdrawal (both P=0.02), with high hostility scores almost reaching significance at P=0.055.

The combined results for serum Histamine (high plus low), percentage of free copper and plasma zinc patients versus controls did not reach significance (see Table 3) however both depressed and elevated levels of histamine did reach significance in relationship to symptom expression. On the diagnostic-based symptom group, a significantly greater proportion of patients with low histamine levels (theoretically reflecting an over-methylating state), did experience more psychotic symptoms than did the control group (n= 7/15, compared with 1/15 controls P=0.050). Similarly, on the MBPRS symptom score, low histamine levels were significantly related to lower than average scores for hyperactivity (p=0.02), distractibility (p=0.01) and high scores for auditory hallucinations (P=0.03). Compared with asymptomatic controls, there were no significant associations found for elevated histamine for DSM IV-R diagnostic groups however significantly higher MBPRS raw symptom scores were obtained for hyperactivity (P=0.03) and distractibility (P=0 03), and significantly lower scores for auditory hallucinations.

A low plasma zinc to free serum copper ratio was found significantly associated with a diagnosis of depression (n=14/14, 100%, P=0.02) and symptom groupings associated with depression (n=14/14, 100%, P=0.02).

After grouping diagnoses into broad symptom-dominant groups for anxiety (n=12), depression of mood (n=14), developmental delay (n=71), psychosis (n=15), somatoform symptoms (n=6) & aggression (n=13), mood swings (“bipolar” n=11), Poisson regression showed that:
Abnormally high histamine levels alone (reflecting under-methylation), were predictively associated with depression (P=0.015), somatoform symptoms (P=0.063) and a number of conditions associated with developmental delay (P=0.059).
High histamine combined with a low homocysteine was associated with aggression (P=0.025).
Either excessively high or low histamine levels were associated with psychotic symptoms (P=0.045) and also aggressive symptoms (P=0.025), when combined with low homocysteine levels.

Anxiety was associated with both abnormally high and abnormally low levels of histamine (reflecting both under-methylation and over-methylation states) whenever either of these states was also combined with a low homocysteine state.

### REFERENCES

Agresti A. (2002) Categorical Data Analysis (2^{nd} Ed). Hoboken N.J. John Wiley and Sons.
Conover W J. (1999) Practical Non-parametric Statistics (PED). Wiley. New York.
Hyman, S E. (2007) Can Neuroscience be integrated into the DSM-V? Nature Reviews/Neuroscience 8: 725-738.
Pfeiffer, C C. et al (1975) Mental and Elemental Nutrients, A Physician's Guide to Nutrition and Health Care. Keats Publishing, Inc, New Haven, Conn.
Van Os J. Hanssen M, Bijl R V. Strauss (1969) revisited: a psychosis continuum in the general population? Schizophrenia Research 2000; 45:11-20.

1. A method for diagnosing in a subject, or predicting the susceptibility of a subject to, a mental or neurodegenerative disorder, the method comprising:
(a) obtaining one or more biological samples from the subject;
(b) determining the levels of two or more biomarkers in the sample, wherein the biomarkers are selected from pyrroles, histamine, methionine adenosyltransferase (MAT) activity, homocysteine, copper and zinc, wherein the pyrroles are urinary pyrroles, kryptopyrroles or HPL; and
(c) comparing the levels of the biomarkers determined in (b) with the levels of said biomarkers from one or more control samples,
wherein abnormal levels of the two or more biomarkers in the sample(s) from the subject compared to the one or more control samples is predictive of susceptibility of the subject to a mental or neurodegenerative disorder,
and wherein an abnormal level of pyrroles comprises elevated pyrrole levels, an abnormal level of homocysteine comprises reduced homocysteine, an abnormal level of MAT activity comprises reduced MAT activity and an abnormal level of copper comprises elevated copper, compared to levels of the corresponding biomarkers in the one or more control samples.

2. The method of claim 1 wherein the biological sample comprises blood, whole blood, blood serum, erythrocytes, leukocytes, saliva, sputum, breath, condensed breath, amniotic fluid, cerebrospinal fluid, urine, or tissue.

3. (canceled)

4. (canceled)

5. The method of claim 1 wherein the control sample is derived from one or more individuals known not to suffer from a mental or neurodegenerative disorder or alternatively known to suffer from a specific, diagnosed mental or neurodegenerative disorder.

6. (canceled)

7. The method of claim 1 wherein the pyrroles are urinary pyrroles, HPL or kryptopyrroles, the histamine is serum histamine, the homocysteine is plasma homocysteine, the copper is free serum copper, the zinc is plasma zinc, or wherein the level of MAT activity is determined by measurement of the levels of one or products of MAT activity or the ratio between said products.

8. (canceled)

9. The method of claim 1 wherein MAT is an MAT isoenzyme.

10. (canceled)

11. The method of claim 7 wherein the products of MAT activity are S-adenosylmethionine and S-adenosylhomocysteine.

12. The method of claim 1 wherein a determination of MAT activity, the levels of one or more products thereof, or the ratio between said products, is made in addition to, or in place of, determination of histamine levels.

13. (canceled)

14. The method of claim 1 wherein the homocysteine is fasting plasma homocysteine.

15. (canceled)

16. The method of claim 7 wherein the percentage free serum copper is calculated from serum copper and serum ceruloplasmin levels.

17. (canceled)

18. The method of claim 1 wherein the ratio of plasma zinc to free serum copper is also determined and an abnormal zinc to copper ratio in a sample from the subject comprises a reduced zinc to copper ratio compared to the ratio in one or more control samples.

19.-23. (canceled)

24. The method of claim 1 wherein the method is used to diagnose the phenotype or endophenotype of a mental or neurodegenerative disorder.

25. The method of claim 1 wherein the method is used to determine or predict the severity and/or disability associated with a mental or neurodegenerative disorder.

26. The method of claim 25 wherein the method comprises the step of correlating the number of abnormal biomarker levels with severity and/or disability scores obtained from a predetermined set of patient norms.

27. The method of claim 1 wherein elevated urinary kryptopyrrole levels in a biological sample derived from a subject are indicative of a mental disorder selected from schizophrenia, bipolar disorder, or developmental delay disorders, or symptoms associated therewith, or susceptibility thereto.

28. The method of claim 1 wherein reduced serum homocysteine levels in a biological sample derived from a subject are indicative of a mental disorder selected from depression, developmental delay disorders or anxiety disorders, or symptoms associated therewith, or susceptibility thereto.

29. The method of claim 1 wherein elevated histamine levels in a biological sample derived from a subject are indicative of a mental disorder selected from depression, or somatoform disorder or symptoms such as distractibility and hyperactivity.

30. The method of claim 1 wherein reduced histamine levels in a biological sample derived from a subject are indicative of a mental disorder selected from psychosis or psychotic symptoms such as auditory hallucinations.

31. The method of claim 18 wherein a low plasma zinc to free serum copper ratio is indicative of depression or symptoms associated therewith, or susceptibility thereto.

32. The method of claim 1 wherein accrual of abnormal levels of three of more of said biomarkers is indicative of central auditory processing disorder, schizophrenia or depression, or symptoms associated therewith, or susceptibility thereto.

33. The method of claim 1 further comprising in step (b) the determination of levels of one or more additional biomarkers as disclosed herein.

34. A method for predicting the onset of a mental or neurodegenerative disorder, or one or more symptoms associated therewith, in a subject, the method comprising:
(a) obtaining one or more biological samples from the subject;
(b) determining the levels of a panel of biomarkers in the sample(s), the biomarkers including pyrroles, histamine, MAT activity, homocysteine, copper and zinc, wherein the pyrroles are urinary pyrroles, kryptopyrroles or HPL; and
(c) comparing the levels of the biomarkers determined in (b) with the levels of said biomarkers from one or more control samples,
wherein abnormal levels of at least three of the biomarkers in the sample(s) from the subject compared to the one or more control samples is predictive of the onset of a mental or neurodegenerative disorder or one or more symptoms associated therewith in the subject,
and wherein an abnormal level of pyrroles comprises elevated pyrrole levels, an abnormal level of homocysteine comprises reduced homocysteine, an abnormal level of MAT activity comprises reduced MAT activity and an abnormal level of copper comprises elevated copper, compared to levels of the corresponding biomarkers in the one or more control samples.

35.-46. (canceled)

47. A method for diagnosing in a subject, or predicting the susceptibility of a subject to, a mental or neurodegenerative disorder, the method comprising:
(a) obtaining a biological sample from the subject;
(b) determining the level of homocysteine or histamine in the sample; and
(c) comparing the level of homocysteine or histamine determined in (b) with the level of homocysteine or histamine from a control sample,
wherein a reduced level of homocysteine or histamine in the sample from the subject compared to that from the control sample is predictive of susceptibility of the subject to a mental or neurodegenerative disorder.

48. The method of claim 47 wherein the level of homocysteine is determined in the sample, and the mental or neurodegenerative disorder is Alzheimer's disease.

49. The method of claim 47 wherein the level of histamine is determined in the sample, and the mental or neurodegenerative disorder is schizophrenia or positive symptoms associated with schizophrenia.
